(19) United States
(12) Patent Application Publication
(10) Pub. No.: US 2019/0337412 A1
(43) Pub. Date: Nov. 7, 2019

(54) ADJUSTABLE SEAT ASSEMBLY AND INPUT FROM A HEALTH CARE PROVIDER

(71) Applicants: Winsen C. Zouzal, Detroit, MI (US); Lear Corporation, Southfield, MI (US)

(72) Inventors: Winsen C. ZOUZAL, Detroit, MI (US); Giuseppe GHISONI, Milano (IT); Gerald PATRICK, Shelby Township, MI (US)

(21) Appl. No.: 15/971,341

(22) Filed: May 4, 2018

(51) Int. Cl. B60N 2/02; A61B 5/11

(52) U.S. Cl. CPC B60N 2/0248; A61B 5/486; A61B 5/1116

(57) ABSTRACT

A method to adjust a seat assembly transmits input indicative of a seating position of an occupant to a health care provider. Input indicative of a prescribed seating position is received from the health care provider. The seat assembly is adjusted to the prescribed seating position. Makes, models and seat assemblies are stored for various vehicles. Ranges of adjustments permitted for the seat assemblies are stored. A selection of a vehicle seat assembly is received. The ranges of adjustments for the selected vehicle seat assembly are outputted. The occupant is evaluated by a medical professional. A prescribed seating position for the occupant is determined. The prescribed seating position is transmitted to a seat assembly controller to adjust the seat assembly.

### TECHNICAL FIELD

Various embodiments relate to adjustable seat assemblies, databases and external input for adjustment of the seat assemblies.

### BACKGROUND

An adjustable seat assembly is disclosed in O'Bannon et al. U.S. Patent Application Publication No. US 2015/0352979 A1, which published to Lear Corporation on Dec. 10, 2015.

### SUMMARY

According to at least one embodiment, a method to adjust a seat assembly transmits input indicative of a seating position of an occupant to a health care provider. Input indicative of a prescribed seating position is received from the health care provider. The seat assembly is adjusted to the prescribed seating position.

According to a further embodiment, the occupant is evaluated by a medical professional. The prescribed seating position is determined for the occupant. The prescribed seating position is transmitted to adjust the seat assembly.

According to another further embodiment, makes, models and seat assemblies are stored for various vehicles. Ranges of adjustments permitted for the seat assemblies are stored. A selection of a vehicle seat assembly is received. The ranges of adjustments for the selected vehicle seat assembly are outputted.

According to another embodiment, a seat assembly controller is programmed to receive input indicative of a seating position of an occupant in a seat assembly. The input indicative of the seating position is transmitted to an external receiver. Input indicative of a prescribed seating position is received. A plurality of actuators in the seat assembly is adjusted to adjust the seating position to the prescribed seating position.

According to another embodiment, a controller external of a vehicle, is programmed to receive input indicative of a seating position of an occupant from a seat assembly controller that is programmed to receive the input indicative of the seating position of the occupant in a seat assembly. The input indicative of the seating position is transmitted to an external receiver. Input indicative of a prescribed seating position is received. A plurality of actuators in the seat assembly is adjusted to adjust the seating position to the prescribed seating position. The external controller displays the seating position upon a display. Input indicative of a prescribed seating position is received. The input indicative of the prescribed seating position is transmitted to the vehicle seat assembly controller.

According to another embodiment, a controller external of a vehicle is programmed to store makes, models and seat assemblies for various vehicles. Ranges of adjustments permitted for the seat assemblies are stored. Selection of a vehicle seat assembly is received from a seat assembly controller that is programmed to receive input indicative of a seating position of an occupant in a seat assembly. The input indicative of the seating position is transmitted to an external receiver. Input indicative of a prescribed seating position is received. A plurality of actuators in the seat assembly is adjusted to adjust the seating position to the prescribed seating position. The external controller outputs the ranges of adjustments for the selected vehicle seat assembly.

According to a further embodiment, a second controller external of the vehicle is programmed to receive input indicative of the seating position of the occupant from the seat assembly controller. The ranges of adjustments for the selected vehicle seat assembly are received. The seating position and the ranges of adjustments for the selected vehicle seat assembly are displayed upon a display. Input indicative of a prescribed seating position is received. The input indicative of the prescribed seating position is transmitted to the vehicle seat assembly controller.

According to another embodiment, an adjustable seat assembly is provided with a seat bottom, and a seat back extending upright adjacent to the seat bottom. A plurality of actuators is supported upon at least one of the seat bottom and the seat back. A controller is in cooperation with the plurality of actuators to adjust a seating position of a seated occupant. The controller is programmed to receive input indicative of the seating position. The input indicative of the seating position is transmitted to an external receiver. Input indicative of a prescribed seating position is received. The plurality of actuators is adjusted to adjust the seating position to the prescribed seating position.

According to a further embodiment, the seat assembly controller is further programmed to store the input indicative of the seating position.

According to an even further embodiment, the seat assembly controller is further programmed to receive input indicative of multiple seating positions. The input indicative of the multiple seating positions is stored.

According to another even further embodiment, the seat assembly controller is further programmed to transmit the input indicative of the multiple seating positions to an external receiver.

According to another embodiment, a method to adjust a seat assembly stores makes, models and seat assemblies for various vehicles. Ranges of adjustments permitted for the seat assemblies are stored. A selection of a vehicle seat assembly is received. The ranges of adjustments for the selected vehicle seat assembly is outputted.

According to a further embodiment, an occupant is evaluated by a medical professional. The ranges of adjustment for the selected vehicle seat assembly are received. A prescribed seating position for the occupant is determined. The prescribed seating position is transmitted to a seat assembly controller to adjust the seat assembly.

According to another embodiment, a controller external of a vehicle is programmed to store makes, models and seat assemblies for various vehicles. Ranges of adjustments permitted for the seat assemblies are stored. Selection of a vehicle seat assembly is received. The ranges of adjustments for the selected vehicle seat assembly are outputted.

According to another embodiment, a method to adjust a seat assembly evaluates an occupant by a medical professional. A prescribed seating position for the occupant is determined. The prescribed seating position is transmitted to a seat assembly controller to adjust the seat assembly.

According to a further embodiment, the occupant is seated upon the seat assembly. The seated occupant upon the seat assembly is evaluated by the medical professional.

According to another further embodiment, ranges of adjustments for the seat assembly are received. The prescribed seating position is determined within the ranges of adjustments.

According to another further embodiment, input indicative of a seating position of the occupant is received. The seating position of the occupant is evaluated by the medical professional. The prescribed seating position is determined for the occupant in response to the evaluated seating position.

According to an even further embodiment, input indicative of multiple seating positions is received. The input indicative of the multiple seating positions is stored.

According to another embodiment, a controller external of a vehicle is programmed to receive input indicative of a seating position of an occupant. The seating position is displayed upon a display. The input indicative of a prescribed seating position is received. The input indicative of the prescribed seating position is transmitted to a vehicle seat assembly controller.

### BRIEF DESCRIPTION OF THE DRAWINGS

FIG. 1 is a front perspective view of a seat assembly according to an embodiment, illustrated partially disassembled;

FIG. 2 is an environmental view of a health care provider with a patient and a display according to an embodiment;

FIG. 3 is a view of a display of a database according to an embodiment;

FIG. 4 is a view of a display of the seat assembly of FIG. 1, illustrating pressure distribution, according to an embodiment;

FIG. 5 is another view of the display of the seat assembly of FIG. 1, illustrating adjustment options for the seat assembly, according to an embodiment;

FIG. 6 is another view of the display of the seat assembly of FIG. 1, illustrating other adjustment options for the seat assembly, according to an embodiment;

FIG. 7 is a view of a display of adjustment settings of the seat assembly of FIG. 1, according to an embodiment;

FIG. 8 is a view of a display of adjustment options of the seat assembly of FIG. 1, according to another embodiment; and

FIG. 9 is another view of the display of adjustment options of FIG. 8.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Back pain and postural issues can be recurring problems that are manageable, if addressed. Unless preventable, these issues should be managed to mitigate chronic pain and disability. Prevention and management involves diet, exercise, maintaining a good posture, visiting a health care professional on a regular basis, and the like. Personal posture management can offer a management tool to seated occupants to manage their health and wellness.

A comfort, posture and wellness seating system for vehicle seat assemblies, provides a visual interface with adjustment hardware organically or inorganically. The system may be employed to properly configure any new or existing seating system. The system can also address specific comfort, posture or preferences, such as thoracic support. The seating system objectifies comfort data and biomechanical knowledge to make the data transferable.

The comfort, posture and wellness seating system integrates anthropometry, bio-mechanics, and historical seating comfort data. The seating system can be employed in original equipment for vehicles or in aftermarket products. Applicable markets include automotive, mass transit, airlines, etc., as well as non-vehicular seating such as office, home, commercial, and public venue seating.

Data collection may be conducted that includes expert positioning of a suitable sample of occupants for optimal comfort or preferred posture by a medical professional. The data collection can be used at specific sites on an ongoing basis if required. The expert input provides a high level of expert comfort, posture and personalized fitting. The data may be based on anthropometry, body pressure distribution (BPD), status of actuators (such as pressure of inflatable air bladders, status of valves or the like), or other data that provides a comfort, posture and biomechanically optimized position of an adjustable vehicle seat assembly. The data is collected in a knowledge base or table for setting adjustments based on categories of data. The knowledge base may be compiled from the expert positioned data and the occupant specific data. The setting adjustments from the knowledge base are utilized for pre-set options in a vehicle seat assembly 20. The setting adjustments can be customized by a user at a controller or display.

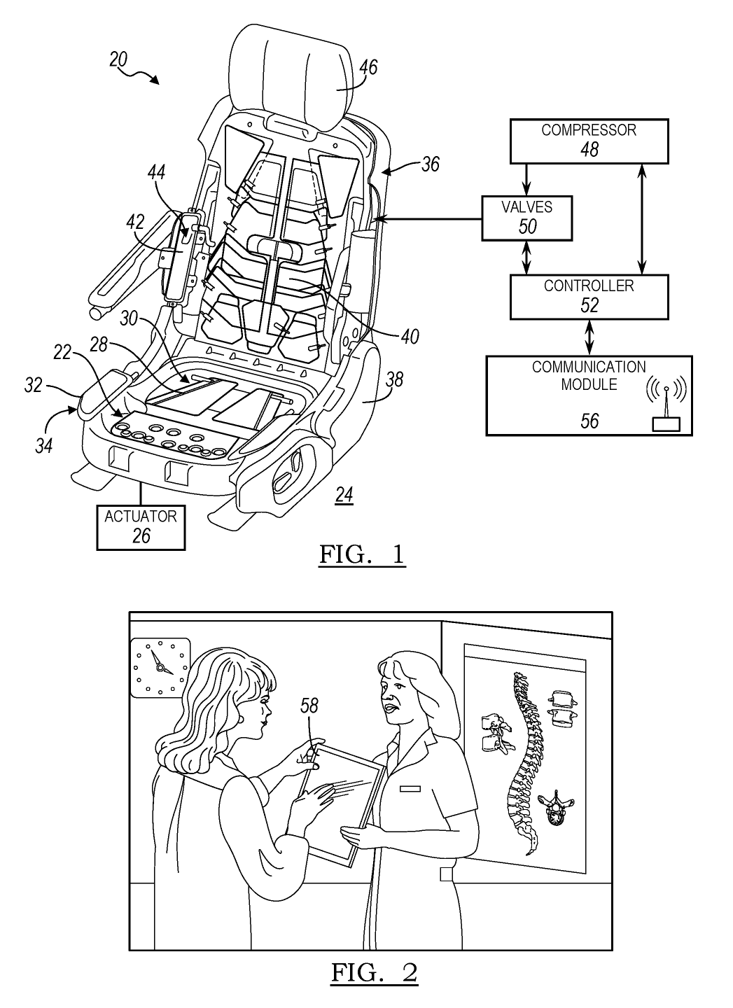

FIG. 1 illustrates the vehicle seat assembly 20 while revealing internal components. The seat assembly 20 includes a seat cushion 22 adapted to be mounted to a floor 24 of a vehicle body. The seat cushion 22 is mounted to the vehicle floor 24 for adjustable tilt about a lateral axis within a limited pivotal range to raise a lower a front of the seat cushion 22. An actuator 26, such as a motor-driven actuator 26 is provided beneath the seat cushion 22 to adjust an angle of the seat cushion 22 relative to the vehicle floor 24 to regulate thigh support. Tilt actuators 26 are known in the art, and the tilt actuator 26 is illustrated schematically in FIG. 1. The seat assembly 20 may also include additional motor-driven actuators 26 for translation in a fore and aft direction and in an up and down direction of the vehicle.

The seat cushion 22 includes a pair of central inflatable air bladder assemblies 28 spaced apart in a sacral region 30 of the seat cushion 22. The seat cushion 22 also includes a pair of bolster air bladder assemblies 32, each oriented within one of a pair of side bolster regions 34 of the seat cushion 22.

The seat assembly 20 includes a seat back 36 pivotally connected to the seat cushion 22 to extend generally upright relative to the seat cushion 22 with a limited range of pivotal movement. Motor-driven pivotal adjustment of the seat back 36 relative to the seat cushion 22 is provided by a seat back recline actuator 38. Recline actuators 38 are also known in the art, and the recline actuator 38 is also illustrated schematically in FIG. 1.

A central air bladder assembly 40 is provided in the seat back 36 within pelvis, lumbar and thoracic regions of the seat back 36. A pair of side bolster air bladder assemblies 42 are each provided within one of a pair of seat back side bolster regions 44. A head restraint 46 is mounted for motor-driven adjustable translation to the seat back 36.

At least one compressor 48 provides a source of air to the seat assembly 20. A plurality of valves 50 receive the compressed air and are controlled by a controller 52 for regulating compressed air into and out of the seat assembly 20. The valves 50 may be provided as a common valve bank that is housed in the seat back 36 or under the seat cushion 22; or the valves 50 may each be provided on each of the air bladders 28, 32, 40, 42. The compressor 48 may be provided in the seat back 36, the seat cushion 22 or concealed within the vehicle body. The controller 52 may be provided in a module under the seat cushion 22, and may be a multifunction controller that also controls other functions in the vehicle.

It is believed that supporting the thoracic region of the spine can reduce forces and support as much as one-third of the upper body mass. By increasing support of the upper body mass, loads are reduced on the muscles, ligaments, and spine and pelvic regions. Decreased load reduces fatigue on these areas of the body. The individual air bladders of the central air bladder assembly 40 are adjustable to provide the appropriate degree of support in the correct location to reduce such loading.

The controller 52 receives the adjustment settings from the pre-set data or from the customized data. The data may be input from one or more interfaces that is/are provided in the vehicle. The interface may be integrated into the vehicle, such as an instrument panel display. The interface may be remote, such as a personal digital assistant (PDA) including phones, tablets and the like. The interface may be provided as a smart device application, wherein users enter relevant information about themselves. The smart phone interface may not require on-site expertise or seat properties. The remote interface permits a user to transport settings to each vehicle, such as personal passenger vehicles, airline seating, rental cars, and the like.

Misalignments of spinal vertebrae and discs may cause irritation to the nervous system and may be an underlying cause to many health problems. Additionally, spinal misalignments can be a contributing factor to a herniated disc, a bulging disc, a facet joint problem, osteoarthritis and spinal stenosis. Sequential adjustment of a seat assembly can enhance posture to minimize spinal misalignments.

Each of the air bladders 28, 32, 40, 42 may include a pressure sensor to detect air pressure in the respective bladder 28, 32, 40, 42. Any pressure sensor is contemplated, such as a pneumatic pressure sensor at the outlet valve of each respective air bladder 28, 32, 40, 42. Pressure can also be sensed by contact pressure sensors disposed in front of or behind some, or all of, the respective air bladders 28, 32, 40, 42 including on a front or rear surface thereof. The contact pressure sensors may include pressure-sensing mats, such as those available by Tekscan®, Inc. of 307 West First Street. South Boston, Mass. 02127-1309, USA.

Although an automated seat adjustment system and method is described, an occupant may elect to obtain personal adjustment settings from a medical practitioner or health care provider as illustrated in FIG. 2. Under this embodiment, the occupant may take their vehicle to their medical practitioner and ask them to adjust the seat assembly 20, to assist with individual postural needs. The personal posture system permits medical practitioners to adjust the functions and bladders 28, 32, 40, 42 of the seat assembly 20, specifically for a patient's individual conditions.

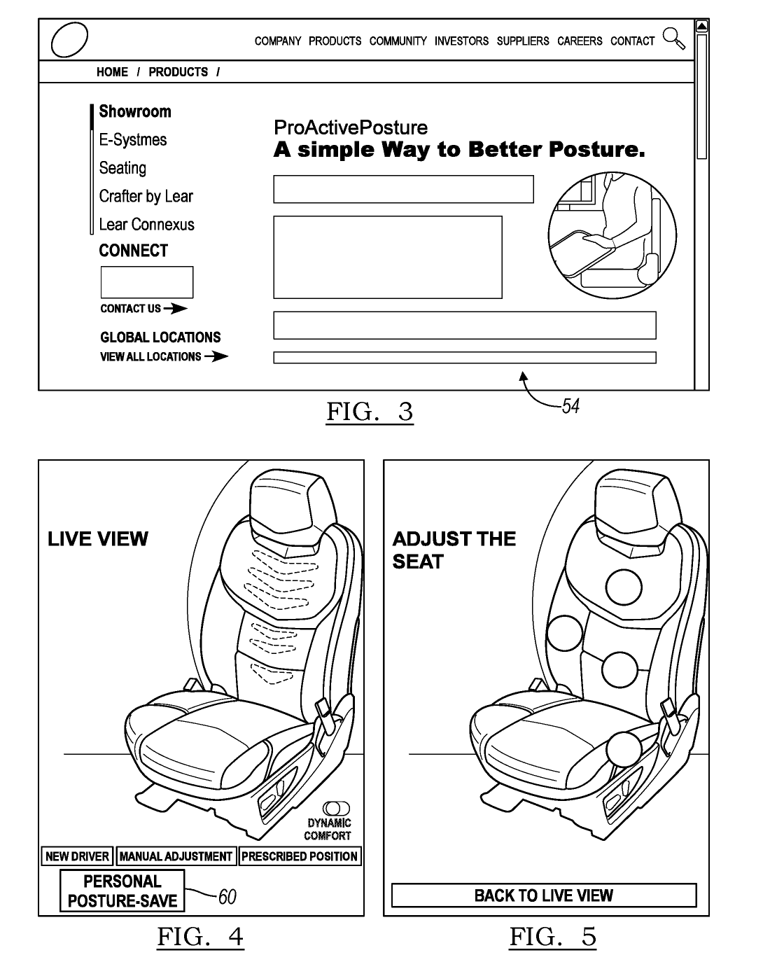

The medical practitioner accesses a database 54, such as a website on the internet, as illustrated in FIG. 3. The database 54 provides general information about the seat assembly 20 with the applicable functionality. The database 54 identifies the locations of the adjustable supports 28, 32, 40, 42 (thoracic, lumbar, cervical, and the like) in the seat assembly 20. The database 54 also offers recommended settings for the seat assembly 20, (such as prescribed, comfort, touring, sport, and the like). The database 54 also identifies the settings of the seat assembly 20 and how to adjust the settings. The database 54 also offers an ability to integrate the seat assembly 20 setting adjustments into therapeutic treatment procedures and to assist physical/functional improvement.

An occupant with the personal posture system may identify the existence of a postural issue, such as discomfort associated with the back, spine and/or cervical disks. For example, the comfort and/or prescribed pre-set modes may not exactly address the support and position requirements of a particular occupant. The occupant may elect to obtain personal posture adjustment after consultation with the medical professional. The occupant may choose to take his or her vehicle directly to a health care provider. The provider may evaluate the occupant, and determine that personalized adjustments to the seat assembly 20 may assist with treatment of the occupant's condition.

Referring to FIG. 1, the controller 52 may be in communication with a communication module 56, such as a vehicle communication module, which may communicate externally via satellite, cellphone or the like. According to one embodiment, the communication module 56 communicates directly with an interface 58 at the health care provider of FIG. 2. According to yet another embodiment, the communication module 56 communicates with the database of FIG. 3. The communication module 56 may transmit input indicative of the seating position from the occupant as measured by the seating sensors. According to at least one embodiment, the valves 50, controller 52 and the communication module 56 are integrated into a common module. Any number of modules are contemplated under various embodiments.

Referring again to FIG. 3, the database 54 may be located on an accessible server that is supported by an automotive dealer, an original equipment manufacturer, an automotive supplier, a third-party service or the like. The database 54 stores the functionality and ranges for various vehicles. The database 54 is searchable by make, model, year, seat position, and the like. The database 54 describes the applicable seating system in the selected vehicle, locations of adjustments, available comfort and prescribed settings, and ranges of potential adjustments.

Referring now to FIG. 4, after the applicable vehicle seat assembly 20 is selected form the database 54, a display of the seat assembly 20 is illustrated. The display is available on the vehicle display. The database 54 of FIG. 3 may also offer the display upon the health care provider interface 58, which includes a transmitter and receiver for communication with the communication module 56 of the seat assembly 20 and the database 54. The display may be common for both the occupant and health care provider interfaces 58 so that the health care provider may make evaluations and adjustments personally or remotely. In FIG. 4, the display illustrates a pressure distribution of the occupant upon the seat assembly 20 as measured by the sensors of the seat assembly 20.

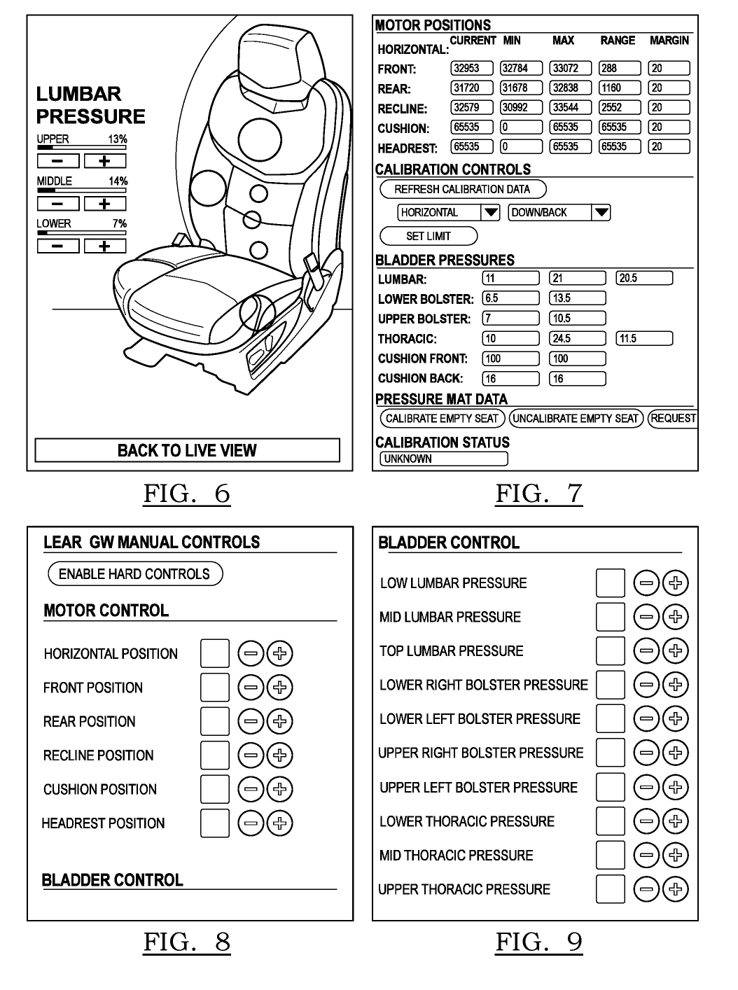

In FIG. 5, the display illustrates the locations for potential adjustments for the seat assembly 20 from the database 54. In FIG. 6, the database 54 illustrates ranges of adjustments of the seat assembly 20 for a selected location upon the display. In FIG. 7, the database 54 illustrates settings for the seat assembly 20 upon the display.

The medical professional of the health care provider can evaluate the patient in person upon the seat assembly 20. The medical professional may also evaluate the patient remotely based upon the information provided from the database. For example, a patient may be evaluated by the medical professional without taking the vehicle to the health care provider.

FIG. 8 illustrates a screen upon the display for manual adjustment of motor-driven actuators of the seat assembly 20. FIG. 9 illustrates a screen upon the display for manual adjustment of various air bladder assemblies within the seat assembly 20. The occupant/patient and health care provider can employ the interface 58 to adjust seat positions and bladders and the corresponding functions to develop a prescribed seating position. Referring again to FIG. 4, a memory button 60 is provided and labeled as “Personal Posture—Save” for saving a prescribed seating position. The prescribed seating position is communicated back to the communication module 56 of the seat assembly 20, and consequently to the controller 52 for adjusting the seat assembly 20 to the prescribed seating position. The prescribed seating position may be communicated directly to the communication module 56, or may be transmitted to the database 54, and then to the communication module 56.

After the seat assembly 20 is adjusted to the prescribed seating position, the occupant and/or the medical professional may access the interface to make additional adjustments to the seating position as desired. The controller 52 may store all adjustments to the seating position, including the prescribed seating position. The controller 52 may also store pressure distributions over time. The history of seating adjustments and pressure distribution is transmitted to the health care provider. Therefore, the medical professional can evaluate the seating position, and pressure distribution relative to the health of the patient to track status and progress. Additionally, the medical professional is relieved of a burden of manually measuring and recording the data. The data is real data measured during use, instead of test data collected under unordinary operating conditions.

The controller 52 may also access other biometric information from the seat assembly 20, such as heart rate, weight, and the like. This biometric data may also be transmitted to the health care provider for evaluation.

While various embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A method to adjust a seat assembly comprising:
transmitting input indicative of a seating position of an occupant to a health care provider;
receiving input indicative of a prescribed seating position from the health care provider; and
adjusting the seat assembly to the prescribed seating position.

2. The method of claim 1 further comprising:
evaluating the occupant by a medical professional;
determining the prescribed seating position for the occupant; and
transmitting the prescribed seating position to adjust the seat assembly.

3. The method of claim 1 further comprising:
storing makes, models and seat assemblies for various vehicles;
storing ranges of adjustments permitted for the seat assemblies;
receiving a selection of a vehicle seat assembly; and
outputting the ranges of adjustments for the selected vehicle seat assembly.

4. A seat assembly controller programmed to:
receive input indicative of the seating position of the occupant in the seat assembly; and
perform the method of claim 1.

5. A controller external of a vehicle, the controller programmed to:
receive input indicative of the seating position of the occupant from the seat assembly controller of claim 4;
display the seating position upon a display;
receive input indicative of a prescribed seating position; and
transmit the input indicative of the prescribed seating position to the vehicle seat assembly controller.

6. A controller external of a vehicle, the controller programmed to:
store makes, models and seat assemblies for various vehicles;
store ranges of adjustments permitted for the seat assemblies;
receive selection of a vehicle seat assembly from the seat assembly controller of claim 4; and
output the ranges of adjustments for the selected vehicle seat assembly.

7. A second controller external of a vehicle, the second controller programmed to:
receive input indicative of the seating position of the occupant from the seat assembly controller of claim 6;
receive the ranges of adjustments for the selected vehicle seat assembly;
display the seating position and the ranges of adjustments for the selected vehicle seat assembly upon a display;
receive input indicative of a prescribed seating position; and
transmit the input indicative of the prescribed seating position to the vehicle seat assembly controller.

8. An adjustable seat assembly comprising:
a seat bottom;
a seat back extending upright adjacent to the seat bottom;
a plurality of actuators supported upon at least one of the seat bottom and the seat back; and
a seat assembly controller according to claim 4, in cooperation with the plurality of actuators to adjust the seating position of the seated occupant.

9. The adjustable seat assembly of claim 8 wherein the seat assembly controller is further programmed to store the input indicative of the seating position.

10. The adjustable seat assembly of claim 9 wherein the seat assembly controller is further programmed to:
receive input indicative of multiple seating positions; and
store the input indicative of the multiple seating positions.

11. The adjustable seat assembly of claim 10 wherein the seat assembly controller is further programmed to transmit the input indicative of the multiple seating positions to an external receiver.

12. A method to adjust a seat assembly comprising:
storing makes, models and seat assemblies for various vehicles;
storing ranges of adjustments permitted for the seat assemblies;
receiving a selection of a vehicle seat assembly; and
outputting the ranges of adjustments for the selected vehicle seat assembly.

13. The method of claim 12 further comprising:
evaluating an occupant by a medical professional;
receiving the ranges of adjustment for the selected vehicle seat assembly;
determining a prescribed seating position for the occupant; and
transmitting the prescribed seating position to a seat assembly controller to adjust the seat assembly.

14. A controller external of a vehicle, the controller programmed to perform the method of claim 12.

15. A method to adjust a seat assembly, the method comprising:
evaluating an occupant by a medical professional;
determining a prescribed seating position for the occupant; and
transmitting the prescribed seating position to a seat assembly controller to adjust the seat assembly.

16. The method to adjust the seat assembly of claim 15 further comprising:
seating the occupant upon the seat assembly; and
evaluating the seated occupant upon the seat assembly by the medical professional.

17. The method to adjust the seat assembly of claim 15 further comprising:
receiving ranges of adjustments for the seat assembly; and
determining the prescribed seating position within the ranges of adjustments.

18. The method to adjust the seat assembly of claim 15 further comprising:
receiving input indicative of a seating position of the occupant;
evaluating the seating position of the occupant by the medical professional; and
determining the prescribed seating position for the occupant in response to the evaluated seating position.

19. The method to adjust the seat assembly of claim 18 further comprising:
receiving input indicative of multiple seating positions; and
storing the input indicative of the multiple seating positions.

20. (canceled)
